SEQUENCE LISTING (16 sequences)

SEQ ID NO: 1 - DNA, 76 bp, Artificial Sequence
  Aptamer
atagtccctggcgtgcttgacagcaacacgaacactgaacgttcttaacaagcctggcagagcaggtacggtgtca

SEQ ID NO: 2 - DNA, 76 bp, Artificial Sequence
  aptamer
atagtccctggcgtgcttccgcgagagttcgacattcccgaaagtacgacgaaagtccagagcaggtacggtgtca

SEQ ID NO: 3 - protein, 25 aa, Artificial Sequence
  template
DPGNVQKAVCHPTAWDLGKGDFRIL

SEQ ID NO: 4 - protein, 25 aa, Artificial Sequence
  Template
EPADGRKVVCHPTAWDLGHGDFRIK

SEQ ID NO: 5 - DNA, 76 bp, Artificial Sequence
  Aptamer
tgacaccgtacctgctctgcaggagcggaccgcgggggttttcttctgggtcggggataagcacgccagggactat

SEQ ID NO: 6 - DNA, 76 bp, Artificial Sequence
  Aptamer
atagtccctggcgtgcttatccccgacccagaagaaaacccccgcggtccgctcctgcagagcaggtacggtgtca

SEQ ID NO: 7 - DNA, 76 bp, Artificial Sequence
  Aptamer
tgacaccgtacctgctctgtgtgagaagggagtttaggctcgtagggctgggatcgttaagcacgccagggactat

SEQ ID NO: 8 - DNA, 76 bp, Artificial Sequence
  Aptamer
atagtccctggcgtgcttaacgatcccagccctacgagcctaaactcccttctcacacagagcaggtacggtgtca

SEQ ID NO: 9 - DNA, 76 bp, Artificial Sequence
  Aptamer
tgacaccgtacctgctctgccaggcttgttaagaacgttcagtgttcgtgttgctgtcaagcacgccagggactat

SEQ ID NO: 10 - DNA, 76 bp, Artificial Sequence
  Aptamer
tgacaccgtacctgctctacctgatgggtgtcctctagctctgccggtagcccgttttaagcacgccagggactat

SEQ ID NO: 11 - DNA, 76 bp, Artificial Sequence
  Aptamer
atagtccctggcgtgcttaaaacgggctaccggcagagctagaggacacccatcaggtagagcaggtacggtgtca

SEQ ID NO: 12 - DNA, 76 bp, Artificial Sequence
  Aptamer
tgacaccgtacctgctctagccatcaccacggagctgggaaactgttgaataagtggcaagcacgccagggactat

SEQ ID NO: 13 - DNA, 76 bp, Artificial Sequence
  Aptamer
atagtccctggcgtgcttgccacttattcaacagtttcccagctccgtggtgatggctagagcaggtacggtgtca

SEQ ID NO: 14 - DNA, 76 bp, Artificial Sequence
  Aptamer
tgacaccgtacctgctcttgggtcgggaatcacttctacggcaatggcttatcggggtaagcacgccagggactat

SEQ ID NO: 15 - DNA, 76 bp, Artificial Sequence
  Aptamer
atagtccctggcgtgcttaccccgataagccattgccgtagaagtgattcccgacccaagagcaggtacggtgtca

SEQ ID NO: 16 - DNA, 76 bp, Artificial Sequence
  Aptamer
tgacaccgtacctgctctggactttcgtcgtactttcgggaatgtcgaactctcgcggaagcacgccagggactat

(19) United States
(12) Patent Application Publication
(10) Pub. No.: US 2025/0304972 A1
(43) Pub. Date: Oct. 2, 2025

(54) COMPOUNDS FOR INHIBITING THE INTERACTION OF SARS-COV2 WITH HUMAN PROTEIN ACE2

(71) Applicants: UNIVERSITA' DEGLI STUDI DI MILANO, Milano (IT); FONDAZIONE ISTITUTO ITALIANO DI TECNOLOGIA, Genova (IT); SCUOLA SUPERIORE DI STUDI UNIVERSITARI E PERFEZIONAMENTO SANT'ANNA, Pisa (IT)

(72) Inventors: Paolo CIANA, Milano (IT); Electra BRUNIALTI, Milano (IT); Alessandro Maria Giovanni VILLA, Milano (IT); Angelo REGGIANI, Genova (IT); Vincenzo LIONETTI, Pisa (IT)

(21) Appl. No.: 18/547,439

(22) PCT Filed: Feb. 22, 2021
(86) PCT No.: PCT/IB2022/051541

§ 371 Date: Aug. 22, 2023

(30) Foreign Application Priority Data
Feb. 22, 2021 (IT) 102021000004007

(51) Int. Cl. C12N 15/115; A61P 31/14; C12N 15/10

(52) U.S. Cl. CPC C12N 15/115; A61P 31/14; C12N 15/1048; C12N 2310/16; C12N 2320/13

(57) ABSTRACT

Novel compounds capable of blocking viral infections sustained by the SARS-Cov2 virus are provided. A method for preventing and/or treating infectious diseases caused by a virus involving administering the novel compounds is also provided.

### CROSS-REFERENCE TO RELATED APPLICATIONS

This application is a National Stage Application of International Patent Application No. PCT/IB2022/051541, having an International Filing Date of Feb. 22, 2022, which claims priority to Italian Application No. 102021000004007, filed Feb. 22, 2021, the entire contents of which are hereby incorporated by reference herein.

### REFERENCE TO AN ELECTRONIC SEQUENCE LISTING

The contents of the electronic sequence listing named “39447-286SEQ.txt”, 4,860 bytes, created on Jan. 24, 2024, is herein incorporated by reference in its entirety.

### TECHNICAL FIELD OF THE INVENTION

The present invention relates to the medical field and, in particular, to preventing and treating viral infections caused by SARS-Cov2.

### BACKGROUND OF THE INVENTION

The SARS-CoV2 virus infects lung cells by using the membrane protein ACE2 (Angiotensin-converting enzyme 2) as access. The interaction with ACE2 occurs through S-glycoprotein (or SPIKE) for which there is a homology with the SPIKE proteins identified on other members of the Orthocoronavirinae subfamily, such as SARS-CoV (e.g., SARS-CoV-2 and variants thereof) and HCoV-NL63; however, there is a significant difference in structure in some of the interaction sites with ACE2, supporting the hypothesis of a different species-specificity of SARS-CoV2 with respect to other coronaviruses.

Severe acute respiratory syndrome coronavirus 2, abbreviated as SARS-CoV-2, is a viral strain belonging to the subgenus Sarbecovirus, of the coronavirus subfamily (Orthocoronavirinae), responsible for diseases ranging from the common cold to more serious diseases such as Middle Eastern respiratory syndrome (MERs) and severe acute respiratory syndrome (SARS). Coronaviruses are a large family of viruses, but only six (229E, NL63, OC43, HKU1, MERS-CoV, SARS-CoV) were previously known for the ability to infect humans; thus SARS-CoV-2 is the seventh. The infection deriving from SARS-CoV-2 is known as COVID-19, acronym for COronaVIrus Disease 19 and the spread thereof has reached the size of a global pandemic today.

The SARS-CoV-2 virus infects target cells using the membrane protein Angiotensin-converting enzyme 2 (ACE2) as access.

The interaction occurs through S-glycoprotein (or SPIKE) which represents a real “hook” used by the virus to couple to the target cells, enter and replicate. The protein spike consists of two components: the subunit S1, a very flexible region containing the protein domain called RBD (receptor-binding domain), through which the virus is capable of recognizing and binding the ACE2 receptor, and the subunit S2 containing a small region called FP, which is the “needle” through which the virus manages to penetrate the target cell.

Once the subunit S1 of the protein spike has bound the ACE2 receptor on the target cell, the subunit S2 changes shape and “sticks” the FP region into the host cell membrane, initiating the invasion process.

ACE2 is an enzyme found on the membranes of the cells of the lungs, arteries, heart, kidneys, and intestines. ACE2 is part of a regulatory complex linked to the Renin-Angiotensin-Aldosterone system (RAAS) and acts as an exopeptidase, i.e., catalyzing the conversion of angiotensin I into the nonapeptide angiotensin (1-9), or angiotensin II into angiotensin. Recently, ACE2 has been found to act as an entry receptor into cells for some coronaviruses, including SARS-Cov-2, such as SARS-CoV (variants included) and HCoV-NL6 due to the high affinity to the protein SPIKE for specific areas of the enzyme. The potency of this spike-ACE2 bond seems to be the main determinant of the great contagiousness of the virus. Lastly, the portion of ACE2 involved in binding to the protein SPIKE is different from the enzyme activity zone.

Aptamers have been developed against SARS-CoV-2 infection as an anti-COVID19 instrument (PMID: 19684916) with the aim of targeting the viral protein Spike.

The publication of Chunyun Sun et al. (“SARS-CoV-2 and SARS-CoV Spike-RBD Structure and Receptor Binding Comparison and Potential Implications on Neutralizing Antibody and Vaccine Development” bioRxiv, 20 Feb. 2020, pages 1-18, XP055812679) reports a study on the residues involved in the interaction between ACE2 and the receptor binding domain (RBD) of the protein Spike and the effect of RDB-specific antibodies on protein Spike neutralization and blocking ACE2 activity.

### SUMMARY OF THE INVENTION

The inventors of the present patent application have designed oligonucleotides, which are surprisingly capable of selectively blocking, in non-covalent mode, the specific protein domains on the ACE2 protein, which recognize and bind the viral portion Spike.

Thereby, the initial Spike-ACE2 interaction is inhibited, thus protecting the target cell from virus aggression and subsequent infection.

Therefore, the invention described in the present patent application is based on an innovative approach; in fact, unlike the traditional approach which tends to neutralize the virus by virtue of vaccination, it protects the target cells by blocking the access of the virus and the replication thereof in the cells themselves.

### BRIEF DESCRIPTION OF THE DRAWINGS

FIG. 1 shows the protein domains of interaction between SPIKE and ACE2. A) interaction between SARS-CoV-2 SPIKE N501 and ACE2 (human) K353: the entry point of the virus into the cell. Docking of the RDB of SPIKE on human ACE2 was performed via the HADDOCK program. The figure shows the best of the possible interactions, calculated by the program, between the two proteins highlighting how in this configuration, the residues N501 and K353 are adjacent and can establish a productive interaction. B) comparison between the ACE2 sequences present in the different mammals shown in the figure.

FIG. 2 shows the structure of human and mouse oligopeptides drawn around lysine residue 353 in humans (A) and histidine (B) in mice. The oligopeptides show a conserved three-dimensional structure around residue 353.

FIG. 3 shows the degree of conservation of the hotspot flanking sequence (K355 in the human sequence, H355 in the murine sequence) between H. sapiens and M. musculus.

FIG. 4 shows the diagram of the SELEX strategy used to identify specific aptamers for the selected ACE2 region. The cycles were repeated ten times.

FIG. 5 shows the sequence diagram (SEQ) of the aptamers identified with the two SELEX procedures using the human peptide (H) or the murine peptide (M). The numbers accompanying the letters H and M refer to the selection cycle where a sequencing protocol (NGS) was applied. The selection with the human peptide was split after the IV cycle (H4) and interrupted in one case at the eighth cycle while it was brought up to cycle 10 in the second “branch”.

FIG. 6 shows the table with the percentages of representativeness of the different sequences for each cycle: only sequencing fragments represented by more than 0.1% were taken into account.

FIG. 7 shows the sequence of all the identified aptamers.

FIG. 8 shows a graph showing the modulation of the interaction between SPIKE-CoV2 and ACE2 induced by treatment with two concentrations (0.20 and 20 μg/mL) of aptamers. STD=Positive control standard provided by the kit.

FIG. 9 shows the graphs with the curves representative of the change in interaction between SPIKE-CoV2 and ACE2 as the concentration of the aptamers measured as a change in absorbance and related to binding in the absence of aptamers (100%) changes. The IC50s were calculated from the curves.

FIG. 10 shows the spatial structures of the complex formed between human ACE2 and aptamers s obtained with bioinformatic docking studies, carried out using High Ambiguity Driven biomolecular DOCKing (HADDOCK) software.

FIGS. 11, 12 and 13 show the results of the assays on the efficacy of the aptamers in preventing infections in cells. In particular, FIG. 12 shows in vivo fluorescence images of HEK293TN-hACE2 cells stained with Alexa546 labelled aptamers. The left panel shows the DAPI fluorescence, the central panel the Alexa 546 fluorescence and the right panel the sum of the two channels identifying the non-membrane bound aptamers. FIG. 13 shows the analysis of ACE2 expression in A549 cells transiently transfected for comparison with an empty vector or expressing hACE2 or mACE2, confirming the expression on the cell surface of the transfected cells.

FIG. 14 shows the results of experiments conducted with lentiviral pseudovirus.

FIG. 15 shows the table showing the activity data of the aptamers on the viral variants.

FIG. 16 shows the experimental data of the two compounds according to the present invention in inhibiting the interaction between the SARS-CoV-2 SPIKE protein and hACE2.

### OBJECT OF THE INVENTION

In a first object, the present patent application describes compounds capable of binding to the human protein ACE2 and inhibiting the interaction between the viral protein SPIKE and the human protein ACE2.

In a second object, such compounds are described for medical use and, in particular, for preventing and/or treating virus infection which exploits ACE2 as a receptor to enter the target cell.

In preferred aspects, such viruses are the SARS-CoV and HCoC-NL63 virus.

In a third object, a pharmaceutical formulation comprising one of the compounds of the invention is described.

In a fourth object, an in vitro method for detecting compounds capable of binding to the human protein ACE2 and inhibiting the interaction between the viral protein SPIKE and the protein ACE2 is described.

In accordance with a fifth object, a method is described for treating a disease comprising the use of the compounds of the invention.

### DETAILED DESCRIPTION OF THE INVENTION

According to a first object, the present invention describes compounds capable of binding to the human protein ACE2 and inhibiting the interaction between the viral protein SPIKE and the human protein ACE2.

In more detail, such compounds are capable of inhibiting the interaction of the residue K353 of the extra-cellular region of the human protein ACE2 with the viral protein SPIKE.

Even more in detail, the compounds of the invention are capable of inhibiting the interaction of the residue K353 of the human protein ACE2 with the residue N501 of the viral protein SPIKE.

In an aspect of the invention, the inhibition of the binding to the viral protein SPIKE, or more in particular to the residue N501 thereof, occurs by binding such compounds to the extracellular region of the human protein ACE2.

According to an aspect of the invention, the compounds described are characterized by a nucleotide sequence: GA(A/C)C; which, being readable in both directions, also comprises the sequence C(A/C)AG.

For the purposes of the present invention, said compounds are represented by the following aptamers:

| SEQ. | | |
|---|---|---|
| ID | | |
| n. | Ref. | Seq |
| 5 | Apt 1 | TGACACCGTACCTGCTCTGCAGGAGCGGACCGCG |
| | | GGGGTTTTCTTCTGGGTCGGGGATAAGCACGCCA |
| | | GGGACTAT |
| 6 | Apt 2 | ATAGTCCCTGGCGTGCTTATCCCCGACCCAGAAG |
| | | AAAACCCCCGCGGTCCGCTCCTGCAGAGCAGGTA |
| | | CGGTGTCA |
| 7 | Apt 3 | TGACACCGTACCTGCTCTGTGTGAGAAGGGAGTT |
| | | TAGGCTCGTAGGGCTGGGATCGTTAAGCACGCCA |
| | | GGGACTAT |
| 8 | Apt 4 | ATAGTCCCTGGCGTGCTTAACGATCCCAGCCCTA |
| | | CGAGCCTAAACTCCCTTCTCACACAGAGCAGGTA |
| | | CGGTGTCA |
| 9 | Apt 5 | TGACACCGTACCTGCTCTGCCAGGCTTGTTAAGA |
| | | ACGTTCAGTGTTCGTGTTGCTGTCAAGCACGCCA |
| | | GGGACTAT |
| 1 | Apt 6 | ATAGTCCCTGGCGTGCTTGACAGCAACACGAACA |
| | | CTGAACGTTCTTAACAAGCCTGGCAGAGCAGGTA |
| | | CGGTGTCA |
| 10 | Apt 7 | TGACACCGTACCTGCTCTACCTGATGGGTGTCCT |
| | | CTAGCTCTGCCGGTAGCCCGTTTTAAGCACGCCA |
| | | GGGACTAT |
| 11 | Apt 8 | ATAGTCCCTGGCGTGCTTAAAACGGGCTACCGGC |
| | | AGAGCTAGAGGACACCCATCAGGTAGAGCAGGTA |
| | | CGGTGTCA |
| 12 | Apt 9 | TGACACCGTACCTGCTCTAGCCATCACCACGGAG |
| | | CTGGGAAACTGTTGAATAAGTGGCAAGCACGCCA |
| | | GGGACTAT |
| 13 | Apt 10 | ATAGTCCCTGGCGTGCTTGCCACTTATTCAACAG |
| | | TTTCCCAGCTCCGTGGTGATGGCTAGAGCAGGTA |
| | | CGGTGTCA |
| 14 | Apt 11 | TGACACCGTACCTGCTCTTGGGTCGGGAATCACT |
| | | TCTACGGCAATGGCTTATCGGGGTAAGCACGCCA |
| | | GGGACTAT |
| 15 | Apt 12 | ATAGTCCCTGGCGTGCTTACCCCGATAAGCCATT |
| | | GCCGTAGAAGTGATTCCCGACCCAAGAGCAGGTA |
| | | CGGTGTCA |
| 16 | Apt 13 | TGACACCGTACCTGCTCTGGACTTTCGTCGTACT |
| | | TTCGGGAATGTCGAACTCTCGCGGAAGCACGCCA |
| | | GGGACTAT |
| 2 | Apt 14 | ATAGTCCCTGGCGTGCTTCCGCGAGAGTTCGACA |
| | | TTCCCGAAAGTACGACGAAAGTCCAGAGCAGGTA |
| | | CGGTGTCA |

According to a preferred aspect, said compounds are preferably represented by the following aptamers:

| | | SEQ. |
|---|---|---|
| | | ID. |
| Ref. | sequence | n. |
| Apt 6 | ATAGTCCCTGGCGTGCTTGACAGCAAC | 1 |
| | ACGAACACTGAACGTTCTTAACAAGCC | |
| | TGGCAGAGCAGGTACGGTGTCA | |
| Apt 14 | ATAGTCCCTGGCGTGCTTCCGCGAGAG | 2 |
| | TTCGACATTCCCGAAAGTACGACGAAA | |
| | GTCCAGAGCAGGTACGGTGTCA | |

In accordance with a second object of the invention, the present patent application describes the above compounds, and in particular the aptamers Apt. 6 and Apt. 14, for medical use.

According to a preferred aspect, the compounds of the invention are described for medical use in preventing and/or treating virus infections which exploit ACE2 as a receptor to enter the target cell.

In preferred aspects, such viruses are viruses of the subfamily Orthocoronavirinae.

In particular, such viruses are SARS-CoV and HCoC-NL63 viruses.

More in particular, such viruses are represented by the SARS-CoV-2 virus and the variants thereof.

Such variants are preferably represented by the variants wt: Wuhan variant, UK: United Kingdom variant (B.1.1.7), SA: South African variant (B.1.53) and BR: Brazilian variant (P.1).

According to a particular aspect of the present invention, the compounds described can be stabilized and/or modified, for example to improve endonuclease resistance, improve pharmacokinetics, pharmacodynamics and/or biodistribution, as well as to allow the achievement of a given target.

Several strategies can be exploited in this respect.

One of these is the modification of the individual bases or of the ribose residues.

For example, a phosphorus-sulfur bond can be introduced by replacing an oxygen with a sulfur atom (phosphorothioate oligonuletide) or, in the ribose residue, a hydroxyl group can be substituted with a boron atom; to these changes there can be added the substitution in the 2′ position with a methoxyl (2′-OMe), methoxyethyl (2′-MOE) or a fluorine (2′-F) residue.

Alternatively, the bases can be modified, for example by introducing residues of 5-methylcytidine, 5-methyluridine (ribothymidine) or by exploiting abasic RNAs (i.e., without any base).

Another possible modification to the bases comprises the use of CET (constrained ethyl bridged nucleic acid), LNA (locked nucleic acid) or ENA (ethylene-bridged nucleic acid).

Another modification can comprise the use of morpholino phosphorodiamidate oligonuletides (PMO) or peptide nucleic acids (PNA) or trycyclo DNA (tcDNA).

Alternatively, the compounds of the invention can be modified at the 5′ and/or 3′ terminal by PEGylation.

In accordance with a third object, the present patent application describes formulations comprising one or more of the compounds of the invention.

In particular, such formulations comprise one or more pharmaceutically acceptable excipients.

Such formulations can be administered to a patient:
at high risk of exposure to the virus and, therefore, of becoming infected, such as: social and health workers, fragile people, the elderly;
with a positive but asymptomatic or paucisymptomatic swab (which has lost his taste and smell, has asthenia and fever), in order to avoid the onset of hyper-inflammation and multiorgan dysfunction;
at a more advanced stage of the disease, where he is no longer treatable by monoclonal antibodies.

According to a fourth object of the invention, a method is described for identifying compounds capable of binding to the human protein ACE2 and of inhibiting the interaction between the viral protein SPIKE and the extra-cellular region of the human protein ACE2.

In a particular aspect, such compounds are capable of inhibiting the interaction between the viral protein SPIKE and the residue K353 of the extracellular region of the human protein ACE2.

In an even more particular aspect, such compounds are capable of binding to the human protein ACE2 around the residue K353 of the extra-cellular region of the human protein ACE2, also referred to as the hotspot.

Even more in particular, such compounds are capable of blocking the binding to the viral protein SPIKE around the amino acid asparagine at position 501.

For the purposes of the present invention, such a method comprises the step of selecting, from an oligonucleotide library, the oligonucleotides capable of binding to a template sequence.

In particular, such a template sequence is selected from:

| | |
|---|---|
| template sequence 1 | DPGNVQKAVCHPTAWDLGKGDERIL |
| template sequence 2 | EPADGRKVVCHPTAWDLGHGDFRIK |

The above template sequences represent further objects of the present invention.

Such a selection can for example be carried out using SELEX (Systematic evolution of ligands by exponential enrichment) technology.

Following the selection step, in vitro efficacy assays can be performed to determine which of the selected aptamers are capable of inhibiting the interaction between the viral protein SPIKE and the human protein ACE2.

Such in vitro efficacy assays can be conducted by appropriate techniques, such as the ELISA technique.

In accordance with a fifth object of the invention, a method is described for treating diseases comprising the use of the compounds of the invention.

In a particular aspect, such diseases are caused by the SARS-CoV and HCoC-NL63 viruses.

In a preferred aspect, such diseases are caused by the SAR-CoV-2 virus, which, depending on the severity, can include diseases ranging from common cold to more severe diseases such as Middle Eastern Respiratory Syndrome (MERs) and Severe Acute Respiratory Syndrome (SARS).

According to an aspect of the present invention, such diseases can also be caused by a variant of the SARS-CoV-2 virus; in particular, said variant is selected from the group comprising: Wuhan variant, United Kingdom variant (B.1.1.7), South African variant (B.1.53) and Brazilian variant (P.1).

As regards the method of the invention, this comprises the step of administering to a patient in need thereof a pharmaceutically effective amount of a compound according to the invention.

According to an aspect of the present invention, the method can also be conducted for the prevention of such diseases.

For the purposes of the present invention, therefore, a patient in need thereof could be a subject:
at high risk of becoming infected, such as: social and health workers, frail people, the elderly;
who has performed a swab for the detection of SARS-CoV-2 infection which was positive but said patient is asymptomatic or paucisymptomatic (who has lost taste and smell, has asthenia and fever), in order to avoid the occurrence of hyper-inflammation and multiorgan dysfunction;
at a more advanced stage of the disease, possibly where he is no longer treatable, for example, by monoclonal antibodies.

The invention will hereafter be further described by virtue of the following experimental section.

### EXPERIMENTAL SECTION

The selection of aptamers capable of blocking the interaction between the “Receptor Binding Domain” (RBD) of the SARS-CoV2 SPIKE protein and the extracellular region of the human protein ACE2 was obtained by proceeding with a method comprising the following three steps:
i) identification of the specific region on the human protein ACE2 essential for virus docking;
ii) identification, through an in vitro selection protocol called Systematic evolution of ligands by exponential enrichment (SELEX), of synthetic DNA oligonucleotides capable of binding to the sequence identified in point (i);
iii) validation of synthetic oligonucleotides using in vitro efficacy tests to evaluate the effective ability of the selected molecules to inhibit the interaction of SARS-CoV2 SPIKE protein with the extracellular region of the human protein ACE2, and the pharmacodynamic parameters describing such pharmacological activity.

In particular, the procedures used are detailed below.

### i) Identification of a Specific Region on the Receptor (the Human Protein ACE2) Essential for Virus Docking.

Specific interaction sites between SPIKE and ACE2—preserved in species susceptible to the virus—promote infection.

In particular, the most critical interaction for virus infectivity is that between the residue N501 of SPIKE and the residue K353 of ACE2, as also confirmed by bioinformatics studies.

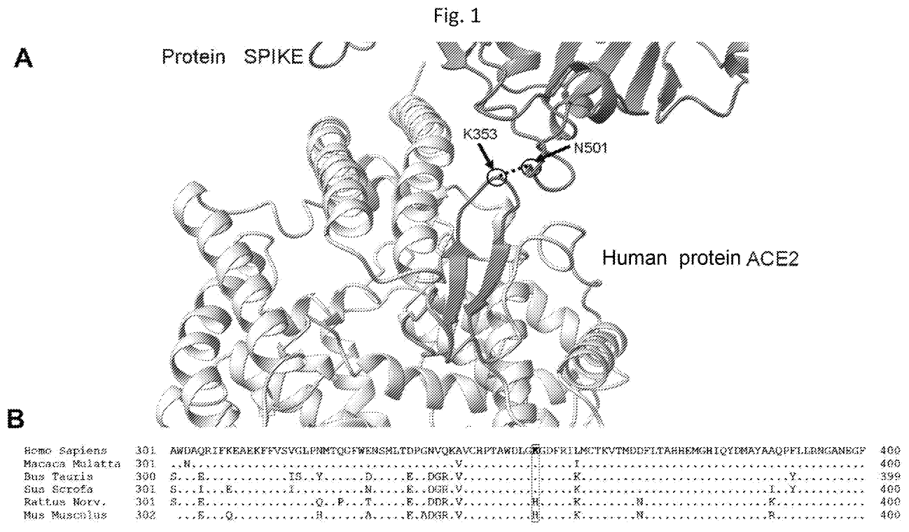

In fact, FIG. 1A shows the interaction protein domains between SARS-CoV-2 SPIKE residue N501 and human protein ACE2 residue K353 and, therefore, the entry point of the virus into the cell. SPIKE's RDB docking on human ACE2 was performed via the HADDOCK program. The figure shows how, in this configuration, the residues N501 and K353 are adjacent and can establish a productive interaction. FIG. 1B shows the comparison between the ACE2 sequences present in the different mammals shown in the figure.

To confirm the importance of the residue K353 in SARS-CoV2 infection, the comparison of the transmembrane domain sequences of the protein ACE2 in different mammals (FIG. 1B) shows an absolute preservation of the sequence itself around the residue K353 in all the species susceptible to the virus, while in mice and rats, species immune to the transmission of SARS-CoV2, a histidine residue is highlighted at position 353 (highlighted in the box, H353). The presence of a histidine (H) in place of lysine (K) leads to the cancellation of the interaction between SPIKE-SARS-CoV2 and ACE2 in the mouse.

In the new variants arising during the pandemic, while not changing the mechanism of interaction, which is always mediated by binding to the residue K353 of ACE2, due to the N501Y mutation (common to all the variants isolated so far) the virus acquires the ability to bind the residue K353 more effectively and also to interact with ACE2 which, as in the case of the mouse or some human polymorphisms, has the residue H353.

Therefore, being able to block the residual hotspot (defined as amino acid 353 in the human sequence) on ACE2 is a possible strategy against new viral variants and also effective on individuals carrying polymorphisms bearing K353.

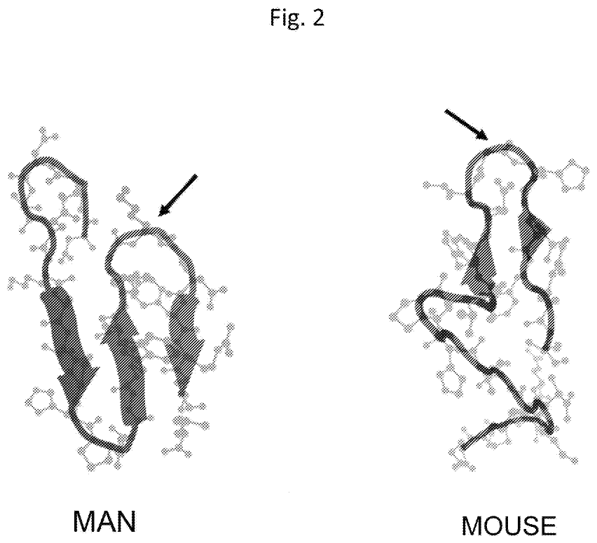

For this purpose, the peptide sequence DPGNVOKAVCHPTAWDLGKGDERIL was identified, which mimics the correct exposure of the lysine residue in the native protein, as indicated by the arrow in FIG. 2A (compare FIG. 2A with FIG. 1A).

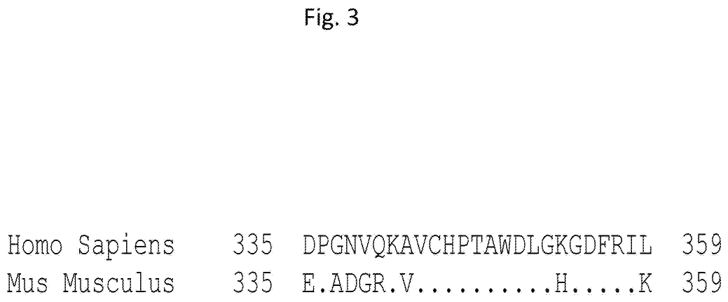

By observing the structure of the same peptide homologous to ACE2 in the mouse EPADGRKVVCHPTAWDLGHGDFRIK, a species refractory to SARS-CoV-2 infection (at least for the most widespread viral variant which has the residue N501 on SPIKE), it can be observed that both the structure (FIG. 2) and the amino acid sequence (FIG. 3) of around the hotspot is very conserved, except for the amino acid of the hotspot (position 353), which is the point of interaction with SPIKE. Based on this observation we thought of using both peptides as baits to identify molecules capable of binding and masking the hotspot regardless of the presence or absence of the amino acid occupying position 353.

### ii) In Vitro Selection of Aptamers Through a Protocol Called Systematic Evolution of Ligands by Exponential Enrichment (SELEX)

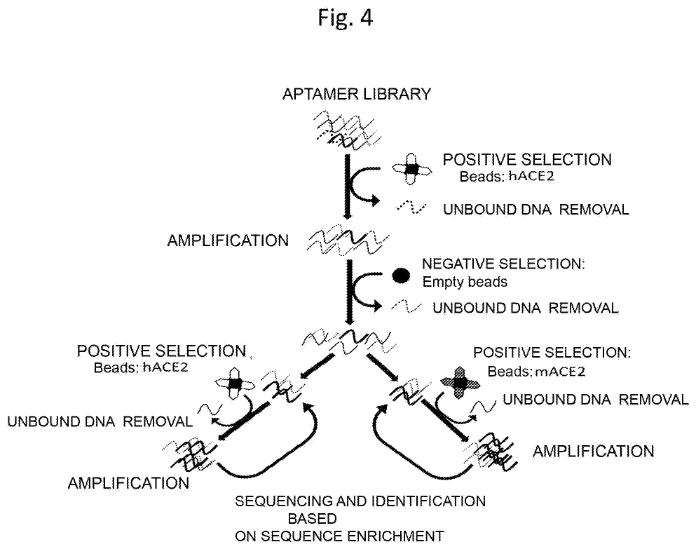

To identify the aptamers capable of highly selectively binding the selected peptides, a SELEX protocol was used (FIG. 4). Thereby, functional oligonucleotides were isolated from a large library of synthetic DNA oligonucleotides (complexity equal to 1024 different sequences) in a very specific manner. Each molecule in this library is a linear oligomer consisting of 76 nucleotides which have a central region formed by a random sequence of 40 nucleotides flanked by two sequences of 18 nucleotides which are constant in all molecules: such sequences form the pairing sites of the two primers necessary for the polymerase chain reaction (PCR) which is an integral part of the SELEX procedure. The selection protocol includes a cycle consisting of: 1) binding oligonucleotides to the target biotinylated peptide immobilized on streptavidin-coated paramagnetic beads, 2) magnetic separation of the peptide-binding oligonucleotides from those which do not bind it, 3) elution and amplification of the binding oligonucleotides and purification of the amplified product (FIG. 4). This cycle was repeated in our SELEX procedure ten times.

After the first selection cycle, a “counter-selection” or “negative elution” cycle was performed (FIG. 4) to remove the aptamers which nonspecifically bound the supports (beads, streptavidin); the product of the “counter-selection” was divided into two equal parts in order to make two parallel selections using two biotinylated peptides: the human peptide (hACE2) and the murine peptide (mACE2). As mentioned in the previous paragraph, the two peptides were drawn on the same protein region and have a very homologous sequence and structure around the hotspot. The two selections were conducted in parallel in order to identify aptamers which were capable of masking the interaction site regardless of the amino acid present in the hotspot.

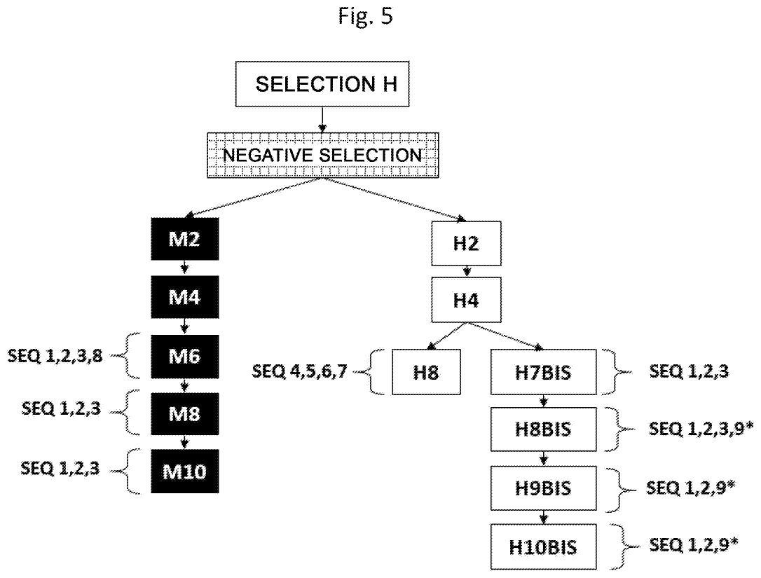

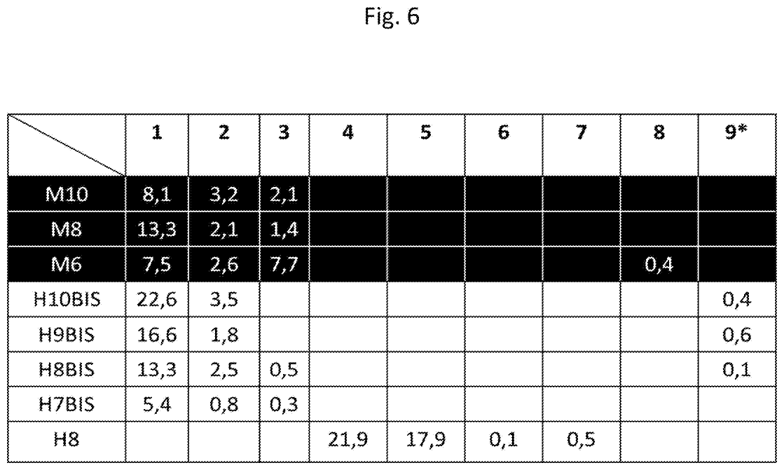

At the end of the tenth cycle, the reaction products were sequenced through Next Generation Sequencing to identify the most represented sequences. The sequencing results showed 8 different sequences from each other (SEQ 1-8) and one sequence, the 9th sequence, which resulted in a mutated variant of SEQ 1 (FIG. 5). The table in FIG. 6 shows the percentages of representativeness of the different sequences for each cycle: only sequencing fragments represented by more than 0.1% were taken into account. The sequences SEQ4-7 were identified only after the VIII cycle because it was not possible to sequence the intermediate cycles due to the scarcity of material produced by the amplification reaction in the previous cycles. The sequence SEQ6 was incomplete and therefore was not considered for subsequent studies.

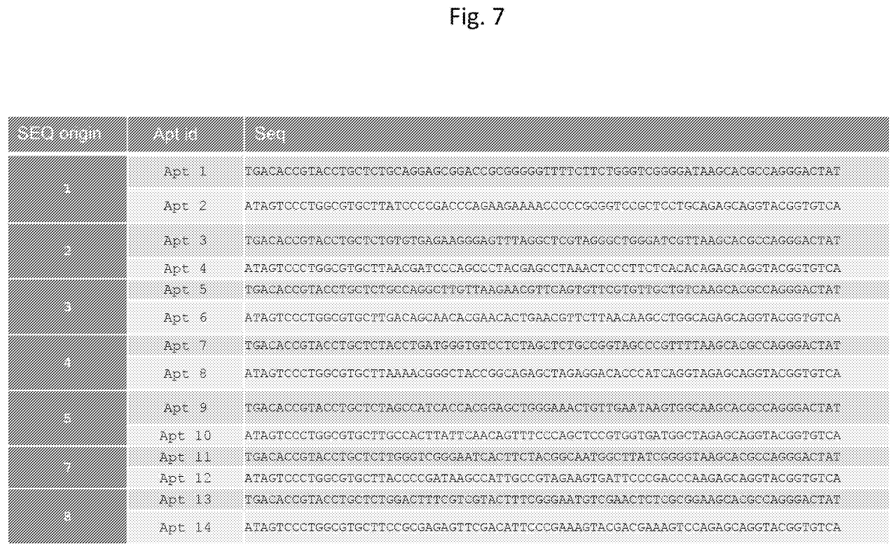

The table in FIG. 7 shows the sequence of all the identified aptamers. For each sequence, two aptamers were obtained considering that the products of the SELEX reaction are double-stranded DNA and it is not possible to know in advance which of the two strands forming the amplified DNA is the one which, when folded, was capable of forming the structure which interacts with the peptide.

iii) In Vitro Efficacy Tests

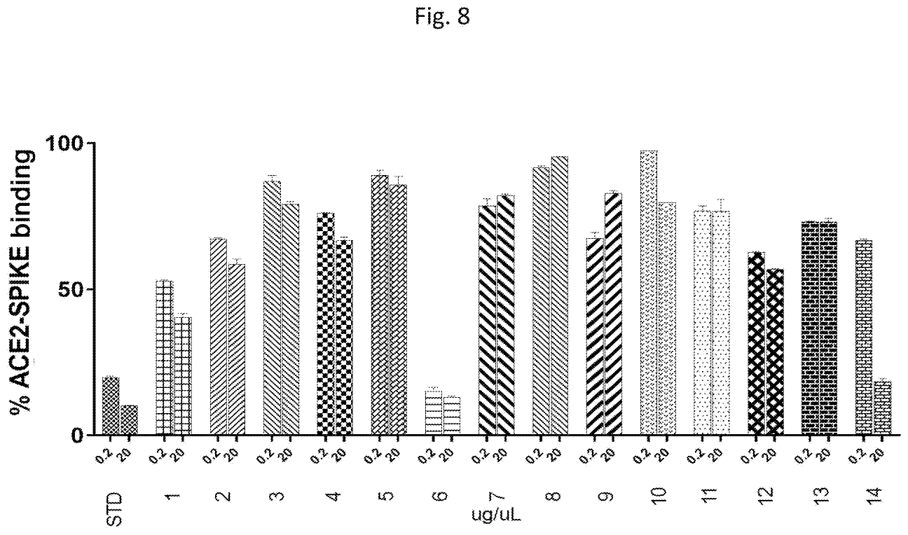

The ability of the 14 identified aptamers to inhibit human SPIKE-CoV-2 and ACE2 binding was tested using a specific kit, manufactured by AcroBiosystems. This test uses a simple colorimetric ELISA system, which allows evaluating in percentage terms—with respect to a control without inhibitor—the link between the RBD of the SPIKE protein of SARS-CoV-2, immobilized on the ELISA plate, and the biotinylated human protein ACE2. A first screening evaluated the effect of two doses of aptamers (0.23 and 23 μg/mL respectively) on the binding between SPIKE-CoV2 and hACE2. The test made it possible to identify two aptamers (Apt. 6 and Apt. 14) capable of inhibiting the interaction between SPIKE and ACE2 in a comparable concentration range with respect to the positive control (STD) provided by the kit manufacturer. FIG. 8 shows the ability of the candidate molecules to inhibit the interaction of the SARS-CoV-2 SPIKE protein, which is expressed as a percentage of an untreated control with inhibitors.

Based on the data obtained with this first general screening, the efficacy study was focused on aptamers 6 and 14. As a negative control, aptamer 1 was used which did not give good results from the point of view of efficacy in interfering with the SPIKE-ACE2 binding.

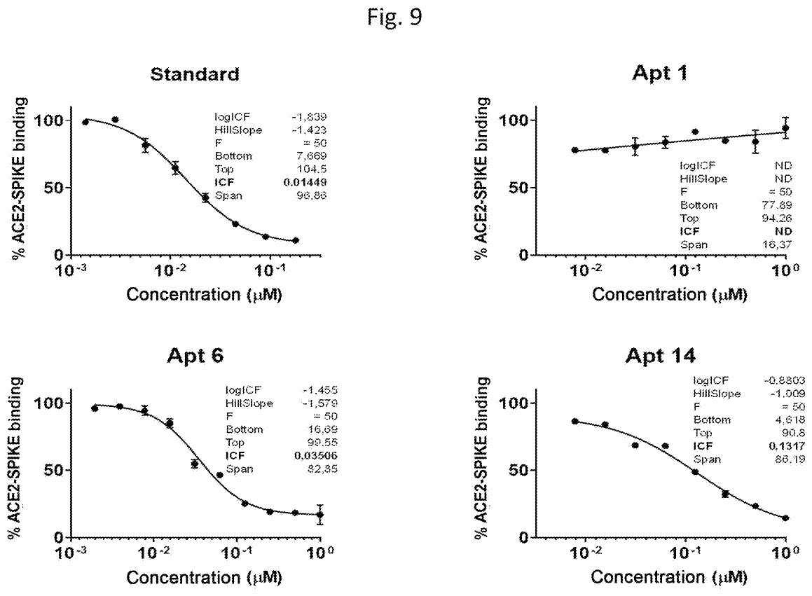

The aptamers were assayed over a wide range of concentrations (pM-μM) and the SPIKE-ACE2 binding inhibition curve made it possible to calculate the IC50, i.e., the concentration of aptamers capable of inhibiting 50% of the interactions between the two proteins. A standard provided by the kit manufacturer was used as a positive control. The results of the in vitro efficacy tests are shown in FIG. 9.

Of the aptamers tested, the most potent in binding ACE2 and inhibiting interaction with the SPIKE-CoV-2 protein was found to be aptamer 6, which appears to have an IC50 of 35 nM; the other candidate aptamer 14 showed an IC50 of 132 mM.

Subsequently, bioinformatics studies were carried out aimed at: i) designing the structures of the aptamers identified through UNA-FOLD software (for the secondary structure) and 3D_DART for the (tertiary structure) and ii) to model aptamer docking in silico using the High Ambiguity Driven biomolecular DOCKing software (HADDOCK).

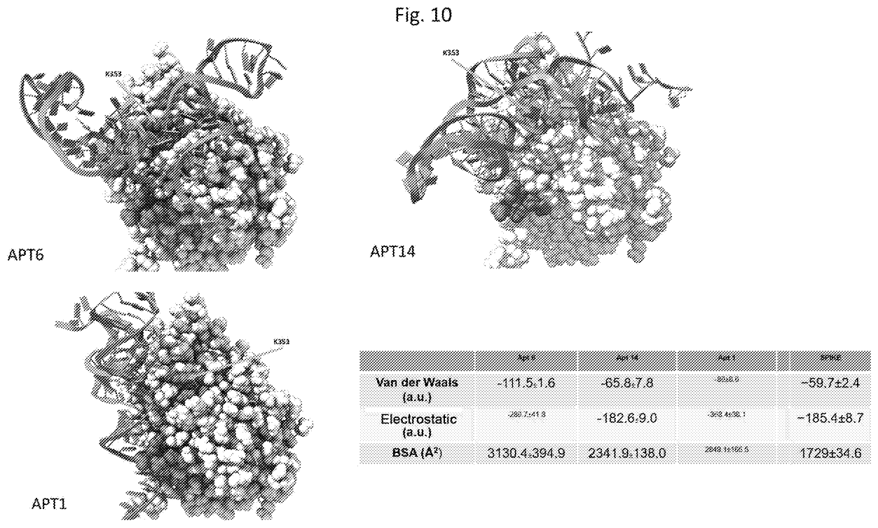

The results of this analysis show the best interaction which these aptamers can have with the human protein ACE2. In particular, it should be noted that while the binding of the aptamers 6 and 14 overlaps precisely with the hotspot, the binding of the aptamer 1 binds at a different position of ACE2 at the tail of the peptide used to select it (FIG. 6). These data are in agreement with what was observed in the efficacy test where only aptamers 6 and 14 are capable of competing with the binding of SPIKE to ACE2, while aptamer 1 is not at all able to block such an interaction. Furthermore, the affinity calculated in silico (table included in FIG. 10) reflects the differences observed between APT6 and APT14 in the efficacy test very well, showing that APT6 is able to displace the binding of ACE2 with SPIKE more effectively than APT14.

Therefore, the two identified aptamers are able to bind and sterically inhibit the binding between SARS-CoV-2 SPIKE and human ACE-2 by masking the residual hotspot (K353) responsible for the binding. The interaction with ACE2 is independent of the hotspot so it can also prevent the binding of SPIKE possessed by different variants of SARS-CoV2 and other SARS viruses which bind ACE2 at the same location.

FIG. 10 shows the spatial structures of the complex formed between human ACE2 and the aptamers: they are the structures calculated by the software as those with lower Wan der Waals and electrostatic energy and therefore provided with greater stability. The aptamer is shown as a grey single-stranded DNA helix plus the arrow indicates the residual hotspot (K353) on ACE2. The shape assumed by the aptamers APT6 and APT14 is positioned exactly on the hotspot, while the aptamer APT1 binds a distal region with respect to such a site. The table included in the figure shows the results of the calculation of the Van der Waals and electrostatic forces and the interaction surface between human ACE2 with the different aptamers and with SPIKE; the results show that APT6 and APT14 form a more stable complex (APT6) or a complex of the same order of magnitude (APT14) as that established by SPIKE. The hACE2-APT1 complex is also energetically favored over the SPIKE complex, however APT1 does not create a steric impediment to SPIKE binding to the hotspot and therefore does not impede the interaction between the two proteins hACE2 and SPIKE.

### iv) Tests for the Efficacy of the Aptamers in Preventing Infections in Cells

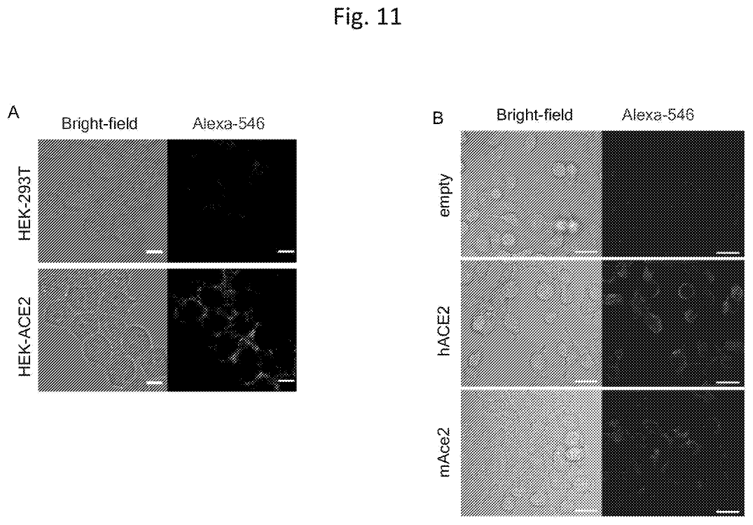

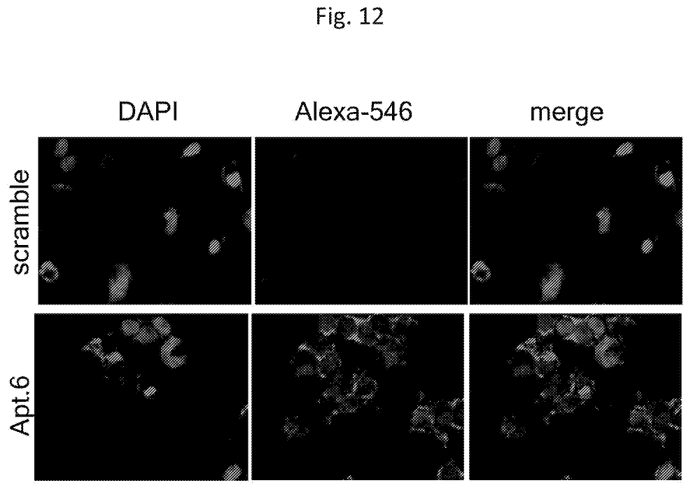

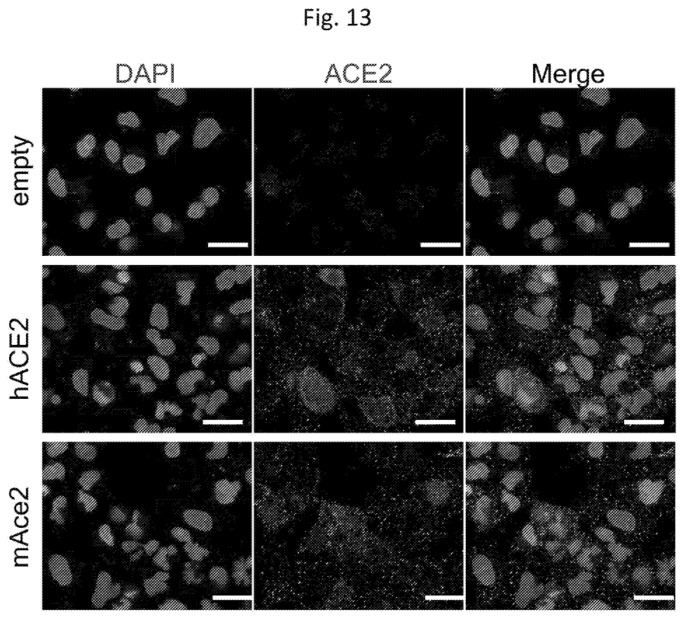

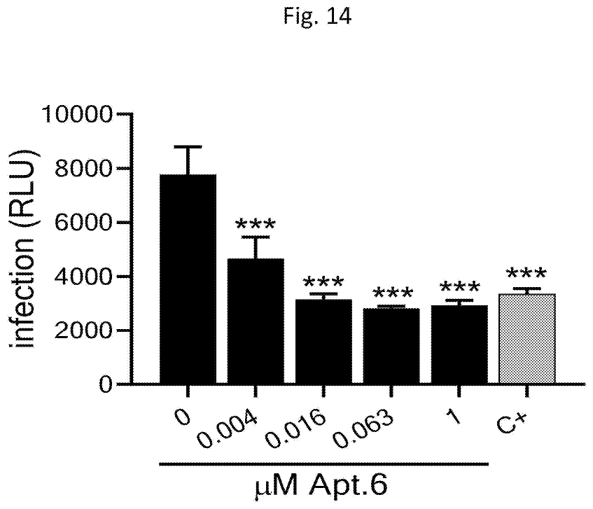

The effectiveness of the aptamers in inhibiting SARS-CoV2 infection is closely linked to the ability thereof to bind the cell receptor thereof on the membrane of the target cells (lung cells). Experiments were thus conducted to demonstrate the ability of aptamer 6 to bind the native receptor, hACE2, on the cell membrane. To this end, renal (HEK293) and pulmonary (A549) cells expressing hACE2 on the membrane thereof and a fluorescent variant of aptamer 6 which allows histological localization were used. The experiments demonstrated the ability of aptamer 6 to bind the receptor on the cell membrane (see FIGS. 11, 12 and 13). The next step was to verify whether binding of aptamer 6 to the receptor on the membrane was also able to prevent the entry of the virus into the cell and therefore the infection thereof. To obtain this demonstration, experiments were conducted with a lentiviral pseudovirus which uses the SARS-CoV2 SPIKE protein to enter the target cell; the genome of the pseudovirus also carries the gene encoding the reporter protein luciferase, which allows simply and quantitatively evaluating (through an enzymatic assay) the ability of the virus to infect the target cell. The experiments with lentiviral pseudovirus unequivocally demonstrated that aptamer 6 is capable of masking the ACE2 receptor and preventing virus entry into the cell (see FIG. 14).

### v) Study of the Activity of the Aptamers on Viral Variants

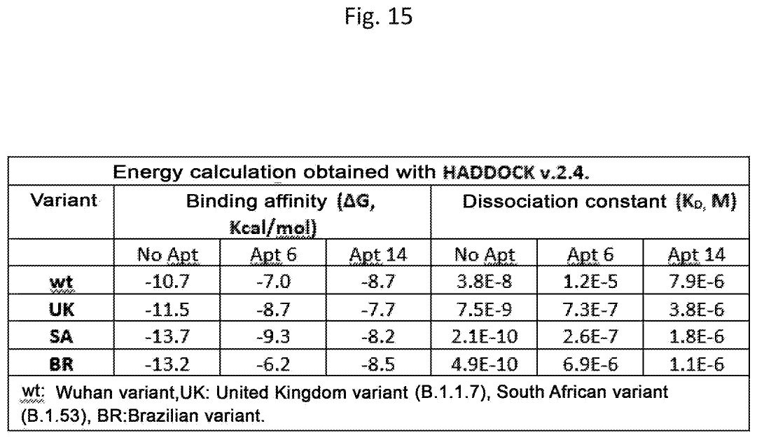

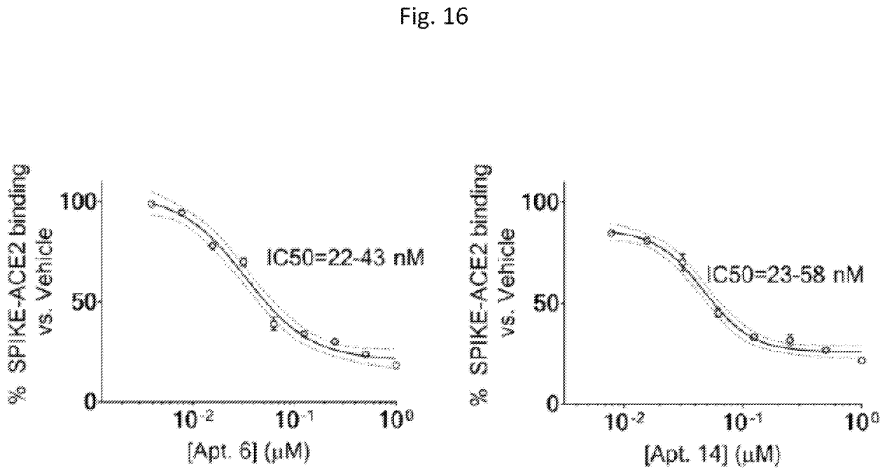

The ability of aptamer 6 to block the interaction of different genetic variants of SARS-CoV2 SPIKE and hACE2 was studied through in vitro assays and through bioinformatic docking simulations. These experiments have shown that the variants wt: Wuhan variant, UK: United Kingdom variant (B.1.1.7), SA: South African variant (B.1.53) and BR: Brazilian variant (P.1) are unable to interact with hACE2 when the aptamer is bound to the receptor (see FIGS. 15 and 16), demonstrating that the aptamers are active at nanomolar concentrations in inhibiting also known variants of SARS-CoV2. In particular, a value of 33 nM was found for aptamer 6 and a value of 47 nM for aptamer 14.

vi) Study of the In Vivo Efficacy of the Activity of Blocking the Entry of COVID-19 into Pneumocytes by the Aptamer

From the foregoing description, the advantages offered by the formulations of the invention will be apparent to the person skilled in the art.

Firstly, having identified ACE2 as the target of the action, the effect is preserved even in the presence of changes in the amino acid sequence of the protein SPIKE and, therefore, in the case of mutations of the virus, which could for example be generated in the future and cause new pandemics.

Furthermore, since a similar mechanism of SARS-CoV infection is conserved in other members of the Orthocoronavirinae subfamily, such as SARS-COV, e.g., SARS-Cov2 and variants thereof) and HCoC-NL63, which have on the surface thereof homologous variants of SPIKE, the spectrum of potential therapeutic applications of the aptamers identified according to the present invention as antiviral drugs will be increased.

Furthermore, the idea that the rate of spread of the contagion and the severity thereof are also due to individual susceptibility or resistance to SARS-CoV2 infection seems to gain credence.

It has been proposed that ACE2 binding domains to SARS SPIKE are involved in the determination of less or greater susceptibility to infection; therefore, the compounds of the present invention are capable of making the target cell “indifferent” to the virus and preventing the spread of infection between cells of different organs.

The innovative approach underlying the present invention could also be useful in improving individual susceptibility.

What is claimed is:

1. A compound capable of binding to human ACE2 protein and inhibiting interaction between a viral spike protein and residue K353 of the extracellular region of the human ACE2 protein.

2. The compound of claim 1, wherein said compound has one of the following sequences:
(SEQ ID NO: 5)
TGACACCGTACCTGCTCTGCAGGAGCGGACCGCGGGGGTTTTCTT
CTGGGTCGGGGATAAGCACGCCAGGGACTAT
(SEQ ID NO: 6)
ATAGTCCCTGGCGTGCTTATCCCCGACCCAGAAGAAAACCCCCGC
GGTCCGCTCCTGCAGAGCAGGTACGGTGTCA
(SEQ ID NO: 7)
TGACACCGTACCTGCTCTGTGTGAGAAGGGAGTTTAGGCTCGTAG
GGCTGGGATCGTTAAGCACGCCAGGGACTAT
(SEQ ID NO: 8)
ATAGTCCCTGGCGTGCTTAACGATCCCAGCCCTACGAGCCTAAAC
TCCCTTCTCACACAGAGCAGGTACGGTGTCA
(SEQ ID NO: 9)
TGACACCGTACCTGCTCTGCCAGGCTTGTTAAGAACGTTCAGTGT
TCGTGTTGCTGTCAAGCACGCCAGGGACTAT
(SEQ ID NO: 1)
ATAGTCCCTGGCGTGCTTGACAGCAACACGAACACTGAACGTTCT
TAACAAGCCTGGCAGAGCAGGTACGGTGTCA
(SEQ ID NO: 10)
TGACACCGTACCTGCTCTACCTGATGGGTGTCCTCTAGCTCTGCC
GGTAGCCCGTTTTAAGCACGCCAGGGACTAT
(SEQ ID NO: 11)
ATAGTCCCTGGCGTGCTTAAAACGGGCTACCGGCAGAGCTAGAGG
ACACCCATCAGGTAGAGCAGGTACGGTGTCA
(SEQ ID NO: 12)
TGACACCGTACCTGCTCTAGCCATCACCACGGAGCTGGGAAACTG
TTGAATAAGTGGCAAGCACGCCAGGGACTAT
(SEQ ID NO: 13)
ATAGTCCCTGGCGTGCTTGCCACTTATTCAACAGTTTCCCAGCTC
CGTGGTGATGGCTAGAGCAGGTACGGTGTCA
(SEQ ID NO: 14)
TGACACCGTACCTGCTCTTGGGTCGGGAATCACTTCTACGGCAAT
GGCTTATCGGGGTAAGCACGCCAGGGACTAT
(SEQ ID NO: 15)
ATAGTCCCTGGCGTGCTTACCCCGATAAGCCATTGCCGTAGAAGT
GATTCCCGACCCAAGAGCAGGTACGGTGTCA
(SEQ ID NO: 16)
TGACACCGTACCTGCTCTGGACTTTCGTCGTACTTTCGGGAATGT
CGAACTCTCGCGGAAGCACGCCAGGGACTAT
(SEQ ID NO: 2)
ATAGTCCCTGGCGTGCTTCCGCGAGAGTTCGACATTCCCGAAAGT
ACGACGAAAGTCCAGAGCAGGTACGGTGTCA.

3. The compound of claim 2, wherein said compound has one of the following sequences:
(SEQ ID NO: 1)
ATAGTCCCTGGCGTGCTTGACAGCAACACGAACACTGAACGTTCT
TAACAAGCCTGGCAGAGCAGGTACGGTGTCA
(SEQ ID NO: 2)
ATAGTCCCTGGCGTGCTTCCGCGAGAGTTCGACATTCCCGAAAGT
ACGACGAAAGTCCAGAGCAGGTACGGTGTCA.

4. (canceled)

5. A method for preventing and/or treating an infectious disease caused by a virus in a subject, said method comprising administering to the subject an effective amount of the compound of claim 1.

6. The method of claim 5, wherein said virus is a virus of the Orthocoronavirinae subfamily, selected from the group consisting of SARS-CoV and HCoC-NL63.

7. The method of claim 6, wherein said virus is the SARS-CoV2 virus or a variant thereof.

8. The method of claim 7, wherein said variant is selected from the group consisting of: Wuhan variant, United Kingdom variant (B.1.1.7), South African variant (B.1.53) and Brazilian variant (P.1).

9. The method of claim 5, wherein said subject is a patient who
is at high risk of becoming infected;
has performed a swab resulting positive but is asymptomatic or paucisymptomatic; or
is at an advanced stage of the infectious disease.

10. A formulation comprising the compound of claim 1.

11. The formulation of claim 10, further comprising one or more pharmaceutically acceptable excipients.

12. An in vitro method for identifying compounds capable of binding to human ACE2 protein and inhibiting interaction between a viral spike protein of a virus and residue K353 of the extracellular region of the human ACE2 protein, the in vitro method comprising selecting, from an oligonucleotide library, the oligonucleotides capable of binding to a template sequence.

13. The in vitro method of claim 12, wherein binding to the viral spike protein of a virus of the Orthocoronavirinae subfamily is blocked.

14. The in vitro method of claim 12, wherein said virus is the SARS-CoV or HCoC-NL63 virus.

15. The in vitro method of claim 12, wherein said virus is the SARS-CoV2 virus or a variant thereof.

16. The in vitro method of claim 15, wherein said variant is selected from the group consisting of: Wuhan variant, United Kingdom variant (B.1.1.7), South African variant (B.1.53) and Brazilian variant (P.1).

17. The in vitro method of claim 12, wherein said template sequence is selected from:
DPGNVQKAVCHPTAWDLGKGDFRIL (SEQ ID NO: 3) and
EPADGRKVVCHPTAWDLGHGDFRIK (SEQ ID NO: 4).

18. The in vitro method of claim 12, wherein identification of the compounds is carried out by SELEX (Systematic evolution of ligands by exponential enrichment) technology.

19. A template sequence selected from:
DPGNVQKAVCHPTAWDLGKGDFRIL (SEQ ID NO: 3) and
EPADGRKVVCHPTAWDLGHGDFRIK (SEQ ID NO: 4).

20-24. (canceled)
